(19) United States
(12) Patent Application Publication
(10) Pub. No.: US 2026/0253589 A1
(43) Pub. Date: Aug. 27, 2026

(54) Systems and Methods for Decoding Speech from Neural Activity

(71) Applicant: The Board of Trustees of the Leland Stanford Junior University, Stanford, CA (US)

(72) Inventors: Krishna V. Shenoy; Jaimie M. Henderson, Redwood City, CA (US); Erin Kunz, Stanford, CA (US); Chaofei Fan, Stanford, CA (US); Francis R. Willett, Palo Alto, CA (US)

(73) Assignee: The Board of Trustees of the Leland Stanford Junior University, Stanford, CA (US)

(21) Appl. No.: 19/102,341

(22) PCT Filed: Aug. 9, 2023
(86) PCT No.: PCT/US2023/071936

§ 371 Date: Feb. 7, 2025

Related U.S. Application Data
(60) Provisional application No. 63/370,920, filed on Aug. 9, 2022.

(51) Int. Cl. G10L 15/24; G06F 3/01; G10L 13/027; G10L 15/06; G10L 15/08; G10L 15/16; G10L 15/187; G10L 25/18; G10L 25/21

(52) U.S. Cl. CPC G10L 15/24; G06F 3/015; G10L 13/027; G10L 15/063; G10L 15/16; G10L 15/187; G10L 25/18; G10L 25/21; G10L 2015/081

(57) ABSTRACT

Systems and methods for decoding speech from neural activity in accordance with embodiments of the invention are illustrated. One embodiment includes a brain-computer interface for decoding intended speech including a microelectrode array, a processor communicatively coupled to the microelectrode array, and a memory, the memory containing a speech decoding application that configures the processor to: receive neural signals from a user's brain recorded by a microelectrode array, where the neural signals comprise action potential spikes, bin the received action potential spikes by time, provide the bins to a recurrent neural network (RNN) to receive a likely phoneme at the time of each provided bin, generate an estimated intended speech using a phoneme decoder provided with the likely phonemes, where the phoneme decoder comprises a language model formatted as a weighted finite-state transducer, and vocalize the estimated intended speech using a loudspeaker communicatively coupled to the brain-computer interface.

### CROSS-REFERENCE TO RELATED APPLICATIONS

The current application is a U.S. National Stage of PCT Patent Application No. PCT/US2023/071936, entitled “Systems and Methods for Decoding Speech from Neural Activity” filed Aug. 9, 2023, which claims the benefit of and priority under 35 U.S.C. § 119(e) to U.S. Provisional Patent Application No. 63/370,920 entitled “Neural Decoding of Attempted Speech” filed Aug. 9, 2022. The disclosure of PCT Patent Application No. PCT/US2023/071936 and U.S. Provisional Patent Application No. 63/370,920 are hereby incorporated by reference in their entireties for all purposes.

### STATEMENT REGARDING FEDERALLY SPONSORED RESEARCH

This invention was made with Government support under contracts DC014034, DC017844, and DC019430 awarded by the National Institutes of Health. The Government has certain rights in the invention.

### FIELD OF THE INVENTION

The present invention generally relates to decoding intended speech from neural activity.

### BACKGROUND

The human brain is a highly complex organ that, among many other functions, generates thought and controls motor function of the body. Different regions of the brain are associated with different functionalities. For example, the motor cortex is involved in the control of voluntary motor functionality. Neural signals in the brain can be recorded using a variety of methods that have different advantages and disadvantages. For example, electroencephalograms (EEGs) are useful for non-invasively measuring average neural activity over a region, with a tradeoff of lower spatial resolution. Implantable microelectrode arrays, such as (but not limited to) the Utah array, are used invasively inside the brain tissue, but can be used to record the activity of a specific or small group of specific neurons with very high spatial resolution. Electrocorticography (ECoG) is an implantable, but slightly less invasive method which involves placing electrodes under the skull on the surface of the brain, which can yield higher spatial resolution than an EEG, but does not rise to the quality of implantable intracranial electrodes.

### SUMMARY OF THE INVENTION

Systems and methods for decoding speech from neural activity in accordance with embodiments of the invention are illustrated. One embodiment includes a brain-computer interface for decoding intended speech including a microelectrode array, a processor communicatively coupled to the microelectrode array, and a memory, the memory containing a speech decoding application that configures the processor to: receive neural signals from a user's brain recorded by a microelectrode array, where the neural signals comprise action potential spikes, bin the received action potential spikes by time, provide the bins to a recurrent neural network (RNN) to receive a likely phoneme at the time of each provided bin, generate an estimated intended speech using a phoneme decoder provided with the likely phonemes, where the phoneme decoder comprises a language model formatted as a weighted finite-state transducer, and vocalize the estimated intended speech using a loudspeaker communicatively coupled to the brain-computer interface.

In another embodiment, the RNN is trained to output an interword demarcator between phonemes that begin and end new words.

In a further embodiment, the RNN is trained using connectionist temporal classification.

In still another embodiment, multiple bins are provided to the RNN at once.

In a still further embodiment, the RNN comprises a unique input layer trained for each day of training data using a softsign activation function.

In yet another embodiment, each bin further comprises high-frequency spectral power features.

In a yet further embodiment, rolling z-scoring is applied to the bins.

In another additional embodiment, the microelectrode array is positioned to record neural activity at a ventral premotor cortex of the user's brain.

In a further additional embodiment, the phoneme decoder traverses the language model using a Viterbi search.

In another embodiment again, the phoneme decoder produces a word lattice using the language model; and wherein the phoneme decoder rescores the word lattice using an n-gram language model such that the best path through the rescored word lattice represents the estimated intended speech.

In a further embodiment again, a method of speech decoding using a brain-computer interface includes recording neural signals from a user's brain using a microelectrode array, where the neural signals are action potential spikes, binning the received action potential spikes by time, providing the bins to a recurrent neural network (RNN) to receive a likely phoneme at the time of each provided bin, generating an estimated intended speech using a phoneme decoder provided with the likely phonemes, where the phoneme decoder includes a language model formatted as a weighted finite-state transducer, and vocalizing the estimated intended speech using a loudspeaker.

In still yet another embodiment, the RNN is trained to output an interword demarcator between phonemes that begin and end new words.

In a still yet further embodiment, the RNN is trained using connectionist temporal classification.

In still another additional embodiment, the method further includes providing multiple bins to the RNN at once.

In a still further additional embodiment, the RNN includes a unique input layer trained for each day of training data using a softsign activation function.

In still another embodiment again, each bin further includes high-frequency spectral power features.

In a still further embodiment again, the method further includes applying rolling z-scoring to the bins.

In yet another additional embodiment, the method further includes positioning the microelectrode array to record neural activity at a ventral premotor cortex of the user's brain.

In a yet further additional embodiment, the method further includes traversing the language model using a Viterbi search.

In yet another embodiment again, the method further includes producing a word lattice using the language model; and rescoring the word lattice using an n-gram language model such that the best path through the rescored word lattice represents the estimated intended speech.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 is a system diagram for a speech decoding system in accordance with an embodiment of the invention.

FIG. 2 is a block diagram for a speech decoder in accordance with an embodiment of the invention.

FIG. 3 is a flow chart for a speech decoding process in accordance with an embodiment of the invention.

FIG. 4 is a graphical depiction of a speech decoding process in accordance with an embodiment of the invention.

### DETAILED DESCRIPTION

Brain-computer interfaces (BCIs) are devices which turn neural activity in the brain into actionable, machine interpretable data. BCIs have many applications from control of prosthetic limbs to enabling users to type on a computer using only thought. A recent advancement in BCI technology has been direct vocalization of intended speech. While typed text strings can be vocalized by a conventional text-to-speech system, it requires the user to in fact type out a text string which can be time consuming. Attempts have been made to directly decode speech from neural activity related to a user speaking. While some success has been found in inferring phonemes from the speech motor area of the brain have been attained, they typically do not perform with enough reliability and/or quickly enough to yield a practical prosthetic speech system for those who have lost the ability physically speak.

Additional attempts have been made to decode handwriting from neural activity. This approach is useful, but for some users it may be more simple to imagine speaking rather than writing. Decoding handwriting from neural activity is described in detail in U.S. Pat. No. 11,640,204 titled “Systems and Methods Decoding Intended Symbols from Neural Activity”, granted May 2, 2023, the disclosure of which is hereby incorporated by reference in its entirety.

Systems and methods described herein utilize a specialized machine learning architecture to decode speech from neural activity associated with speech. In many embodiments, the neural activity arises from the ventral premotor cortex (Brodmann Area 6v) where neural activity is highly separable between movements, however similar methods may be applied to other neural signals that arise from other brain areas that are similarly rich in speech information. In many embodiments, a specific recurrent neural network (RNN) architecture is used which is designed to enhance precision and accuracy in decoding speech.

### Speech Decoding Systems

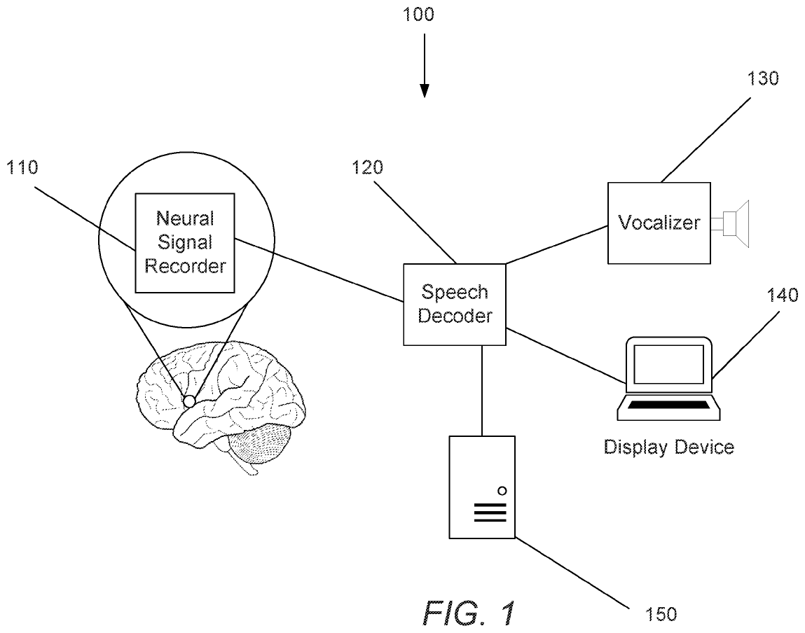

Speech decoding systems can obtain neural signals from a brain using neural signal recorders, and decode the signals into speech. The decoded speech in turn can be vocalized to restore communication to the user. Turning now to FIG. 1, a system architecture for a speech decoding system in accordance with an embodiment of the invention is illustrated.

Speech decoding system 100 includes a neural signal recorder 110. In numerous embodiments, neural signal recorders are implantable microelectrode arrays such as (but not limited to) Utah arrays. The neural signal recorder can include transmission circuitry and/or any other circuitry required to obtain and transmit the neural signals. In many embodiments, the neural signal recorder is implanted into or sufficiently adjacent to ventral premotor cortex. However, as one of ordinary skill in the art can appreciate, systems and methods described herein can implant the neural signal recorder into a number of different regions of the brain including (but not limited to) other motor regions, and focus signal acquisition and subsequent processing based on signals generated from that particular region. For example, instead of focusing on handwriting, similar systems and methods could focus on imagined movement of a leg in a particular fashion to produce similar results.

A speech decoder 120 is in communication with the neural signal recorder. In numerous embodiments, speech decoders are implemented using computer systems including (but not limited to) personal computers, server systems, cell phones, laptops, tablet computers, and/or any other computing device as appropriate to the requirements of specific applications of embodiments of the invention. The speech decoder is capable of performing speech decoding processes for interpreting the acquired neural signals and effecting the appropriate commands.

In many embodiments, the speech decoder is connected to output devices which can be the subject of any of a number of different commands, including (but not limited to) loudspeaker 130, display device 140, and computer system 150. In numerous embodiments, loudspeakers can be used to read out text as speech, or provide other audio feedback to a user or the user's audience. In various embodiments, the text generated by the user can be used to control display devices or other computer systems by forming commands. However as can be readily appreciated, any number of different computing systems can be used as an output device depending on the particular needs of the user and available set of commands.

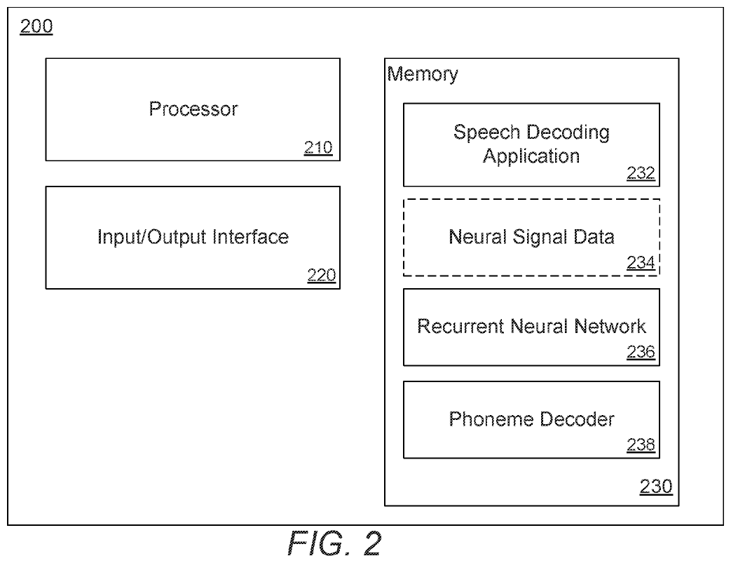

Speech decoders, for example, can be constructed using any of a number of different computing devices. A block diagram for a speech decoder in accordance with an embodiment of the invention is further illustrated in FIG. 2. Speech decoder 200 includes a processor 210. Processors can be any number of one or more types of logic processing circuits including (but not limited to) central processing units (CPUs), graphics processing units (GPUs), field-programmable gate arrays (FPGAs), application-specific integrated circuits (ASICs), and/or any other logic circuit capable of carrying out speech decoding processes as appropriate to the requirements of specific applications of embodiments of the invention.

The speech decoder 200 further includes an input/output (I/O) interface 220. In numerous embodiments, I/O interfaces are capable of obtaining data from neural signal recorders. In various embodiments, I/O interfaces are capable of communicating with output devices and/or other computing devices. The speech decoder 200 further includes a memory 230. The memory 230 contains a speech decoding application 232. The speech decoding application is capable of directing at least the processor to perform various speech decoding processes such as (but not limited to) those described herein. In numerous embodiments, the speech decoding application directs output devices to perform various commands.

In numerous embodiments, at various stages of operation the memory 230 contains neural signal data 324. Neural signal data is data describing neuron activity in a user's brain recorded by the neural signal recorder. In many embodiments, the neural signal data reflects action potentials of individual or a small grouping of neurons (often referred to as “spikes”) recorded using an electrode of an implanted microelectrode array. In a variety of embodiments, the neural signal data describes various spikes recorded at various different electrodes. The memory 230 also contains a recurrent neural network 236 which is trained to predict phonemes from neural signal data, and a phoneme decoder 238 which is configured to produce likely words from strings of phonemes.

While particular system architectures and speech decoders are discussed above with respect to FIGS. 1 and 2, any number of different architectures and speech decoders can be used as appropriate to the requirements of specific applications of embodiments of the invention. For example, in numerous embodiments, a speech decoding system may only have one output device, or various components may be wirelessly connected. As can be readily appreciated, many different implementations can be utilized without departing from the scope or spirit of the invention. Speech decoding processes are discussed in further detail below.

### Speech Decoding Processes

Speech decoding processes can be used to translate brain activity of a user into phonemes, and subsequently into text strings of words which can then be read out. In numerous embodiments, an RNN is trained to convert a time-series of neural activity into phoneme probabilities. In various embodiments, the probability of an inter-word “silence” token and a “blank” token are also viable outputs. The blank token, in particular, can be used in conjunction with a connectionist temporal classification training procedure for the RNN. The end of a series of decoded phonemes is indicated by the silence token which creates a word constructed of phonemes. By demarcating words using the silence token, an increase in performance is achieved as compared to decoding phonemes only. These phoneme word constructs represent discrete word sounds, but not any particular word. The series of phoneme representations of works are decoded further into sentences using a phoneme decoder constructed using a large-vocabulary language model. This constitutes a collapse of the phoneme word constructs into a single word. The resulting sentence can then be vocalized.

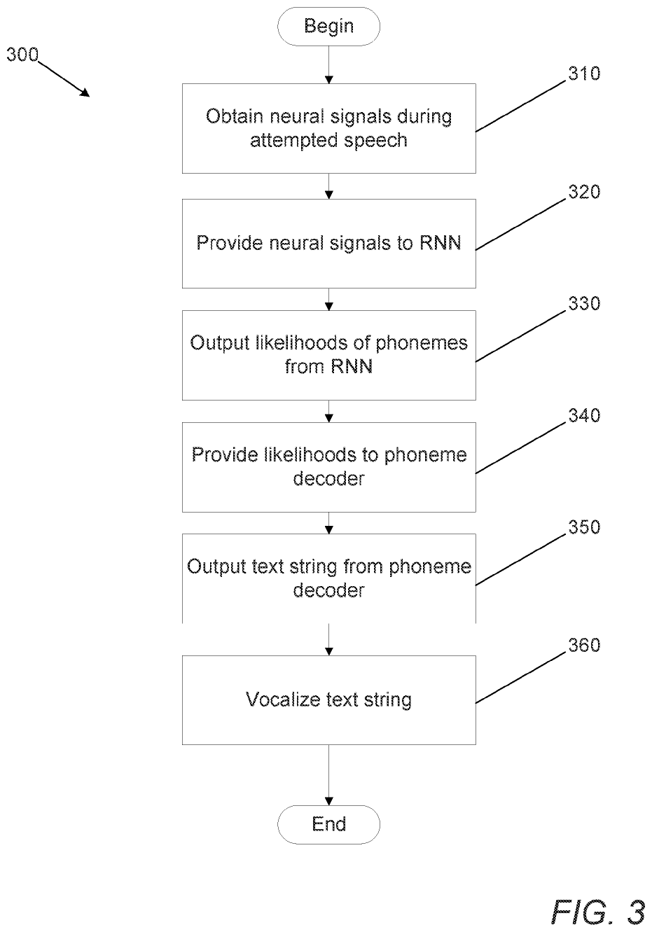

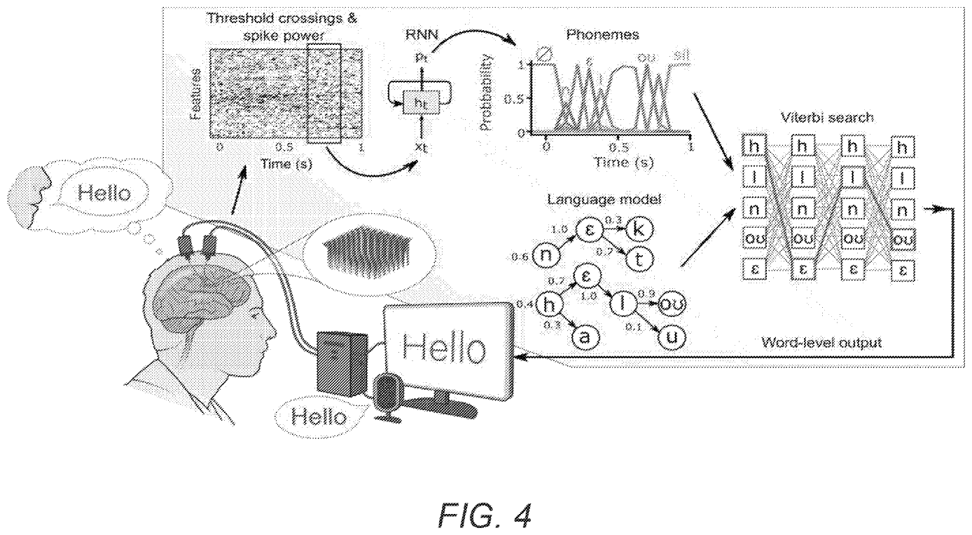

Flow chart for speech decoding in accordance with an embodiment of the invention is illustrated in FIG. 3. Process 300 includes obtaining (310) neural signals from the user's brain using a microelectrode array while the user attempts (or imagines physically) vocalizing natural speech. It is important to note that the user does not need to physically move during this process, and therefore those who cannot physically move a portion of their body can still utilize the systems and methods described herein. In numerous embodiments, the microelectrode array is implanted in the user's brain at the ventral premotor cortex. The neural signal data is provided (320) to an RNN that outputs (330) likelihoods of phonemes associated with the natural speech the user attempted. The phonemes are provided (340) to a phoneme decoder which outputs (350) a word or series of words based on the received phonemes and the most likely sentence intended to be vocalized by the user. The resulting sentences are then vocalized (360) using a loudspeaker. In numerous embodiments, the sentences can be used to control connected devices as commands via natural language processing or as pre-defined command phrases. This process is graphically represented in accordance with an embodiment of the invention in FIG. 4. While phoneme-based decoding has been discussed in previous works, the particular implementations of the RNN and phoneme decoder provide significant boosts to accuracy and precision, as well as processing speed. Experimental use has yielded unconstrained sentence speech decoding from a large vocabulary at a rate of 62 words per minute with an error rate below 25%. A discussion of the RNN is followed by a discussion of the phoneme decoder.

### Speech Decoding RNNs

A core problem for speech decoding is that users may not be able to physically produce intelligible speech. This marks gathering ground truth labels of what phonemes are being spoken extremely difficult if not impossible. This means it is very difficult to apply conventional supervised training techniques to train an RNN. To address this problem, the Connectionist Temporal Classification (CTC) loss function can be used to train the RNN to output a sequence of symbols (phonemes) given an unlabeled time series input. Using the CTC loss function results in an RNN that is trained to output a time series of phoneme probabilities (with an extra “blank” token probability). As noted above, the time series of phoneme probabilities can then be use to infer a sequence of underlying words using a phoneme decoder by simply emitting the phoneme of maximum probability at each time step (while taking care to omit repeats and time steps where “blank” is the maximum probability).

The input to the RNN is neural signal data that is collected using an implanted microelectrode array. In numerous embodiments, the neural signal data is preprocessed by temporally binning and/or temporally smoothing detected spikes on each electrode in the microelectrode array. In many embodiments, the neural signals are analog filtered and digitized. In some embodiments, the analog filter is from 0.3 Hz to 7.5 kHz, and the filtered signals are digitized at 30 KHz at 250 nV resolution. A common average reference filter can be applied to the digitized signals to subtract the average signal across the microelectrode array from every electrode in order to reduce common mode noise. A digital bandpass filter from approximately 250 Hz to 3000 Hz can then be applied. Threshold crossings for each electrode can be performed and the threshold crossing times binned. In many embodiments, the threshold crossing is placed at −4.5×RMS for each electrode, where RMS is the electrode-specific root mean square of the voltage time series recorded for that electrode. In numerous embodiments the temporal binning window is between 10 ms and 300 ms. However, different binning windows can be used based on the user's individual brain. In many embodiments, the temporal bin is 20 ms. The bins are “z-scored” (mean-subtracted and divided by the standard deviation), and causally smoothed by convolving with a Gaussian kernel. Of note is that each brain is highly idiosyncratic, and many parameters described above and elsewhere can be tuned to produce better results for an individual user. Each bin constitutes a neural population time series referred to as xₜ.

In numerous embodiments, the RNN is specifically a 5 layer, stacked gated recurrent unit RNN. In numerous embodiments, one layer is a day-specific input layer that consists of an affine transformation applied to the feature vector followed by a softsign activation function, rather than a purely linear layer. This can enable more adaptable decoding given the drift in neural activity across days. This is formalized as \tilde{x}ₜ=softsign (Wxₜ+b). Here, \tilde{x}ₜ is the day-transformed input vector at timestep t, Wᵢ is a 256×256 matrix and bᵢ is a 256×1 bias vector for day i. The softsign function is applied element-wise to the resultant vector

$$( where softsign ( x ) = \frac{x}{\vert x \vert + 1} ) .$$

Wᵢ and bᵢ are optimized simultaneously along with all other RNN parameters, and dropout is applied both prior to and after the softsign during training.

Rolling z-scoring can further be used to account for neural non-stationaries that accrue across time. Rolling windows can be established of a predetermined length (e.g. 1-10 minutes), can be established. A weighted average of the prior window's mean estimate and the score for the first several sentences of the instant window (e.g. 5-15 sentences) is taken:

$${u}_{i} = \frac{11 - i}{10} * {u}_{prev} + \frac{i - 1}{10} * {u}_{curr} ,$$

Here, using the 10 first sentences in the new window, uᵢ is the mean used to z-score sentence I, uₚᵣₑᵥ is the prior window's mean estimate, and u_{curr} is the mean collected across all sentences in the instant window. After the first several sentences are collected, the previous window's mean is no longer incorporated. In various embodiments, the standard deviation is updated in the same way as the mean.

In various embodiments, artificial noise can be added to the neural features to regularize the RNN. At each time step, white noise can be directly added to the input feature vectors, which improves generalization. Artificial constant offsets can be added to the means of neural features to mark the RNN more robust to non-stationaries. The white noise and constant offset noise can be combined together to transform the input vector in the following way:

$${x}_{t}^{′} = {x}_{t} + {ϵ}_{t} + ϕ ,$$

where ϵₜ is a white noise vector unique to each timestep and φ is a constant offset vector. Other methods for addressing neural drift and associated non-stationary issues are discussed in PCT Patent Application No. PCT/US2023/070758, titled “Systems and Methods for Unsupervised Calibration of Brain-Computer Interfaces”, filed Jul. 21, 2023, the disclosure of which is hereby incorporated by reference in its entirety.

In numerous embodiments, the RNN is trained using a quadratic learning rate schedule which increases performance relative to a linear decay learning rate. Further, bins of data can be stacked together and fed into the RNN one chunk at a time, e.g. kernel size=14 bins, stride=4 bins, or the like, which can increase performance compared to feeding a single bin at a time. Incorporating at least some of the above yields increased performance overall as seen by experimental results. The phoneme decoder is discussed in further detail below.

### Phoneme Decoders

Phoneme decoders as discussed herein take sets of phoneme probabilities and translate them into words and/or sentences. In numerous embodiments, an n-gram language model is used such as (but not limited to) Kaldi, and populated using a large corpus of natural text in the target language. The language model is converted into a weighted finite-state transducer (WFST), which is a finite-state acceptor in which each transition has an input symbol, an output symbol, and a weight. A path through the WFST takes a sequence of input symbols and emits a sequence of output symbols. The WFST is constructed as: T∘L∘G, where: ∘ denotes composition; G is the grammar WFST that encodes legal sequences of words and their probabilities based on the n-gram language model; L is the lexicon WFST that encodes what phonemes are contained in each legal word; and T is the token WFST that maps a sequence of RNN output labels to a single phoneme. In many embodiments, T contains all phonemes plus the CTC blank symbol. In various embodiments, each legal word in L has the silence token appended. In various embodiments, the probability of the silence token is approximately 0.9.

The phoneme decoder runs an approximate Viterbi search (beam search) on the WFST representation of the language model to find the most likely sequence of words. In various embodiments, instead of outputting a decoded sentence a word lattice is output instead, which is a directed graph where each node is a word and the edge between nodes encodes the transition probability between words. An unpruned n-gram language model can be used to rescore the word lattice, after which the best path through the lattice represents the decoded sentence.

Although specific systems and methods for decoding speech from neural activity are discussed above, many different system architectures and methods can be implemented in accordance with many different embodiments of the invention. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A brain-computer interface for decoding intended speech, comprising:
a microelectrode array;
a processor communicatively coupled to the microelectrode array; and
a memory, the memory containing a speech decoding application that configures the processor to:
receive neural signals from a user's brain recorded by a microelectrode array, where the neural signals comprise action potential spikes;
bin the received action potential spikes by time;
provide the bins to a recurrent neural network (RNN) to receive a likely phoneme at the time of each provided bin;
generate an estimated intended speech using a phoneme decoder provided with the likely phonemes, where the phoneme decoder comprises a language model formatted as a weighted finite-state transducer; and
vocalize the estimated intended speech using a loudspeaker communicatively coupled to the brain-computer interface.

2. The brain-computer interface of claim 1, wherein the RNN is trained to output an interword demarcator between phonemes that begin and end new words.

3. The brain-computer interface of claim 1, wherein the RNN is trained using connectionist temporal classification.

4. The brain-computer interface of claim 1, wherein multiple bins are provided to the RNN at once.

5. The brain-computer interface of claim 1, wherein the RNN comprises a unique input layer trained for each day of training data using a softsign activation function.

6. The brain-computer interface of claim 1, wherein each bin further comprises high-frequency spectral power features.

7. The brain-computer interface of claim 1, wherein rolling z-scoring is applied to the bins.

8. The brain-computer interface of claim 1, wherein the microelectrode array is positioned to record neural activity at a ventral premotor cortex of the user's brain.

9. The brain-computer interface of claim 1, wherein the phoneme decoder traverses the language model using a Viterbi search.

10. The brain-computer interface of claim 1, wherein the phoneme decoder produces a word lattice using the language model; and wherein the phoneme decoder rescores the word lattice using an n-gram language model such that the best path through the rescored word lattice represents the estimated intended speech.

11. A method of speech decoding using a brain-computer interface, comprising:
recording neural signals from a user's brain using a microelectrode array, where the neural signals comprise action potential spikes;
binning the received action potential spikes by time;
providing the bins to a recurrent neural network (RNN) to receive a likely phoneme at the time of each provided bin;
generating an estimated intended speech using a phoneme decoder provided with the likely phonemes, where the phoneme decoder comprises a language model formatted as a weighted finite-state transducer; and
vocalizing the estimated intended speech using a loudspeaker.

12. The method of speech decoding using a brain-computer interface of claim 11, wherein the RNN is trained to output an interword demarcator between phonemes that begin and end new words.

13. The brain-computer interface of claim 1, wherein the RNN is trained using connectionist temporal classification.

14. The method of speech decoding using a brain-computer interface of claim 11, further comprising providing multiple bins to the RNN at once.

15. The method of speech decoding using a brain-computer interface of claim 11, wherein the RNN comprises a unique input layer trained for each day of training data using a softsign activation function.

16. The method of speech decoding using a brain-computer interface of claim 11, wherein each bin further comprises high-frequency spectral power features.

17. The method of speech decoding using a brain-computer interface of claim 11, further comprising applying rolling z-scoring to the bins.

18. The method of speech decoding using a brain-computer interface of claim 11, further comprising positioning the microelectrode array to record neural activity at a ventral premotor cortex of the user's brain.

19. The method of speech decoding using a brain-computer interface of claim 11, further comprising traversing the language model using a Viterbi search.

20. The method of speech decoding using a brain-computer interface of claim 11, further comprising producing a word lattice using the language model; and rescoring the word lattice using an n-gram language model such that the best path through the rescored word lattice represents the estimated intended speech.
